(19) United States
(12) Patent Application Publication
(10) Pub. No.: US 2008/0200752 A1
(43) Pub. Date: Aug. 21, 2008

(54) Method for intrauterine embryo transfer and relevant device

(76) Inventor: Pieraldo Inaudi, Florence (IT)

Correspondence Address: THE WEBB LAW FIRM, P.C., 700 KOPPERS BUILDING, 436 SEVENTH AVENUE, PITTSBURGH, PA, 15219, US

(21) Appl. No.: 11/706,445

(22) Filed: Feb. 15, 2007

(51) Int. Cl. A61B 17/435

(52) U.S. Cl. 600/34

(57) ABSTRACT

The invention relates to method and a device for the intrauterine embryo transfer, according to a procedure in which the embryos are aspirated from a culture medium for being ejected into the uterine cavity, wherein the aspiration and the release of the embryos is realized using a catheter having a substantially constant inner diameter in which is inserted a relevant plunger.

### FIELD OF THE INVENTION

The present invention relates to a method for intrauterine embryo transfer and a relevant device for actuating said method.

### BACKGROUND OF THE INVENTION

Over recent years, there have been many efforts to increase the success rate of Assisted Reproduction Technologies. In spite of this, little attention has been addressed to embryo transfer itself, which remains an important rate-limiting step of the IVF (In Vitro Fertilization) procedure. Indeed, even if the ET (Embryo Transfer) is performed in well-standardized conditions, the percentage of embryos found outside the cavity after transfer is too high (range 15-45%), and approx. 30% of all IVF failures can be attributed to the embryo transfer procedure.

The IVF cycle starts with the ovarian stimulation, continues with the ovum pick-up and laboratory procedures and ends with the intrauterine embryo transfer. This last, being at the end of the whole procedure, becomes the most important limiting step of pregnancy rate. The embryo transfer procedure is usually performed in a blind or, if ultrasound guided, in a partially blind way and even if the pregnancy rate is influenced by several factors like the number, quality and stage of development of the embryos, by the receptivity of the uterus, by the instrumentations used (ultrasound, catheter) and by the ability and specific experience of the operators, there is a general agreement on the fact that an easy embryo transfer (short time, no blood, no contractions) has more probability of success than a difficult one. The ability of physician can limit the number of difficult transfer but also the collection of some patient's data, such as the distance between the external os and the uterine fundus and the uterine angle, can help the physician to accomplish the transfer procedure more easily and in less time. On that basis, the embryo transfer procedure should be limited to a gentle introduction of the speculum to expose the cervix, followed by delicate removal of cervical mucus, the catheter loading, insertion in the uterus and, after a gentle release of the embryos in the uterine cavity, slow withdrawal. If any complication occurs at any time, altering the simple and rapid sequence of the above-mentioned steps as a consequence, the success rate could be proportionally reduced. Several factors having a negative impact on the embryo transfer success, have been described as for example the risk of expulsion of the embryos from the uterine cavity. Studies performed on mock transfers using methylene blue demonstrated that the blue dye was present at the cervical os in 20-50% of the cases. One of the reasons for embryo expulsion is uterine contractility, which is mainly induced by increased oxytocin secretion during uterine manipulation. Also, an inadequate loading of embryos at too high a volume may produce a reflux of the medium and the consequent expulsion of embryos from the uterine cavity. The reduction in loading volume is actually associated with a marked increase in pregnancy and implantation rate. Another risk is the possible aspiration of embryos into the catheter or outside the cavity.

The choice of the type of catheter is still a very debatable point. A soft catheter is probably better than a stiff one because it is less traumatic for the endometrium. In effect, the results concerning the Tomcat catheter are unexpected because of the higher percentage of endometrium disruption probably due to the particularly stiff material employed. It is likely that the simplicity of the catheter and the specific experience of operators, plays an important role. Actually, the quality of the embryo transfer depends on the ability of the clinician and the biologist as well as on the catheter employed. As far as the first point is concerned, there are no significant differences in pregnancy rates if the embryo transfer is performed by skilled physicians or nurses, but good results are obtained only when at least 50 embryo transfers have been performed. Moreover, the pregnancy rates among physicians vary between 13 and 37% depending both on the ovarian stimulation and embryo transfer. In effect, only the dexterity and the skill of the operator are able to reduce the risks of embryo loss and it is possible to reduce intra-operator variability only if very experienced operators are involved. This means that it is difficult to maintain the same level of performance from one transfer to another and between the operators; in other words, the embryo transfer procedure remains of a poor standard and with a high risk of error. We believe that it is quite unacceptable that such an important step, which can greatly influence the success of the IVF cycle, remains still burdened by a number of negative, poorly controlled variables.

The type of catheter is rated as the third most important variable for the success of the cycle and several studies, even if not conclusive, have been performed to establish which of the catheters offers the best performance.

### SUMMARY OF THE INVENTION

The present invention relates to the use of a new procedure which utilizes an embryo transfer catheter having some new features, the main one being that it requires no syringe for embryo aspiration and release.

In particular, according to the invention, the transfer is executed using a catheter which is constituted by a hollow cylindrical body, having a substantially constant inner diameter and in which is inserted a plunger which allows a constant flux of the liquid to be transferred, without pressure difference values.

### BRIEF DESCRIPTION OF THE DRAWINGS

A non-limiting embodiment of the present invention will be described by way of example with reference to the accompanying drawing, in which:

FIG. 1 is a schematic lateral view of a possible embodiment of a catheter according to the present invention;

FIG. 2 is a schematic lateral view of a possible embodiment of a plunger for the catheter of FIG. 1.

### DESCRIPTION OF A PREFERRED EMBODIMENT

The present invention has been tested during a study, in which 150 IVF-ET cycles were performed in 150 couples affected by primary or secondary infertility of tubal (37%), male (33%), unexplained (17%), or mixed (13%) origin and 2.5-8.0 years of duration. Exclusion criteria were: age of more than 40 years, high basal FSH (Follicle Stimulating Hormone) concentrations, uterine fibroids, systemic diseases. Due to restriction under the present Italian law on Assisted Procreation, it isn't permitted to produce more than the number of embryos necessary for a sole and simultaneous transfer with a maximum of three embryos. As consequence, in this study, the number of mature oocytes inseminated was corresponding to the number of embryos required by the patients. Only ICSI (Intra Cytoplasmic Sperm Injection) cycles were performed. All the women were pre-treated with a GnRH (Gonadotropin-Releasing Hormone) analogue starting on the 23^{rd} day of their menstrual cycle. After the next menstruation the condition of down-regulation was confirmed if an endometrial thickness of <3 mm and a follicle with a diameter of <8 mm were present. Ovarian stimulation was then started by subcutaneous administration of recombinant FSH (Puregon®, Organon) in doses varying between 100 and 250 IU/day depending on the age of the woman and on the presence of PCOS (Polycystic Ovarian Syndrome). Usually no more then 150 IU was used as starting dose in young women with PCOS. Follicle growth was monitored by ultrasound scans performed every 2-3 days starting on the sixth day of stimulation and hCG (human Chorionic Gonadotropin, in this case: Gonasi, AMSA, Rome, Italy) was administered when at least 2 follicles had reached 18 mm in diameter, while the FSH administration was suspended. At the time of the embryo transfer, i.e. approx 72 hours after collecting the oocytes, the patients were divided in two groups following an alternate allocation in order to have a similar distribution of infertility causes. In the first group, the new catheter was used and in the second group the Tomcat catheter was employed as reference catheter, having been used for 15 years in the hospital in which the study has been performed. Fifty mg/day i.m. progesterone (Prontogest, AMSA, Rome, Italy) were given daily for at least two weeks, starting on the day after the embryo transfer. With reference to the figures of the attached drawings, the new catheter 1, in a preferred but not limitative embodiment, is composed of a rigid proximal tube 2, a semi-rigid medial tube 3 and a soft distal portion 4.

The rigid proximal tube 2 has an external diameter (ED) of 6 Fr (2.00 mm) and a length L2 of about 110 mm.

The semi-rigid medial tube 3 has an ED of 4 Fr (1.33 mm) and a length L3 of about 140 mm.

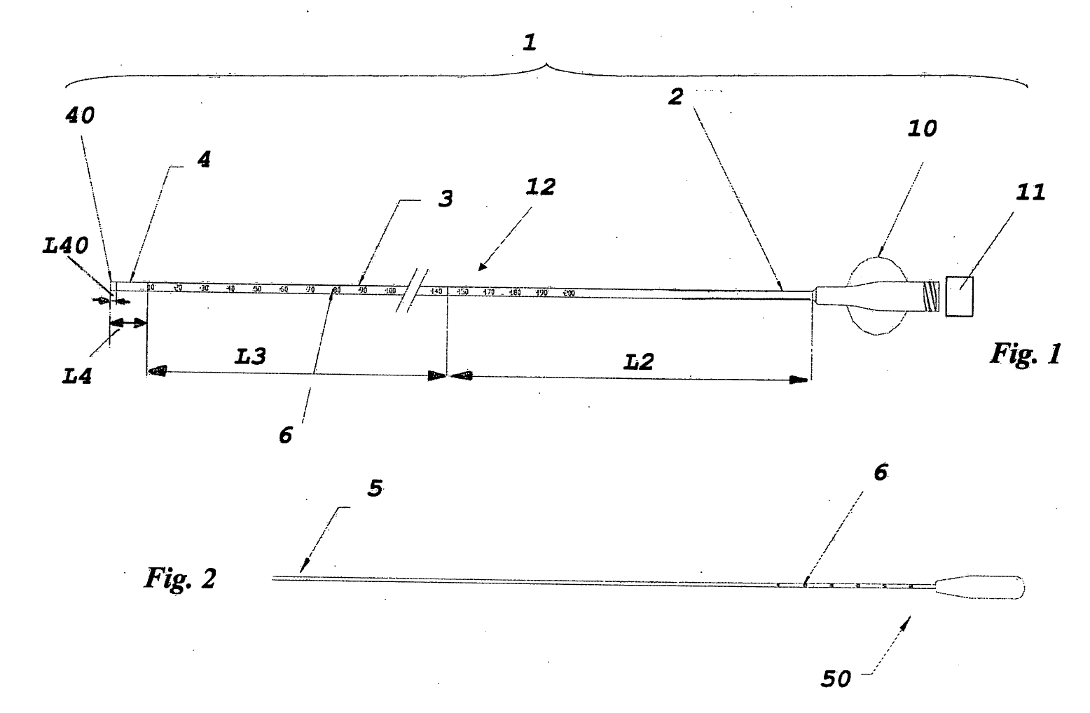

The soft distal portion 4 is about 10 mm long (L4 in FIG. 1), with ED 4 Fr (1.33 mm) and is provided with an echogenic tip 40 of about 2 mm length (L40 in FIG. 1).

The total length of the catheter 1 is substantially comprised between 180 and 320 mm; the inner diameter of the catheter is constant and comprised between 0.1 and 1.0 mm.

According to the embodiment shown in the drawings, the preferred total length of the catheter 1 is about 260 mm and the inner diameter is about 0.4 mm.

Ink marks 6 (i.e. millimeter's marks) are placed on the proximal 2 and central 3 part of the catheter 1. Said marks 6 can be placed also on the proximal part of a relevant plunger 5, shown in FIG. 2. The plunger 5 is inside the catheter 1 and there is no need for a connected insulin syringe, as necessary in the known state of the art. The catheter can be made of polypropylene material and toxicological tests have been done.

Moreover, the catheter 1 is provided with a female connector finger grip 10, provided with a luer lock and with a screwed end on which can be fixed a relevant screwed cap 11. On the catheter can be fitted a stopper element or ring mark 12 which can be fixed in a determined position along the catheter 1. The suitable position of the ring mark 12 on the catheter can be determined by hysterometric measure for controlling the penetration of the same catheter in the uterus.

The plunger 5 is provided with a relevant plunger finger grip 50.

During the tests executed, the embryo transfers were all performed by two experienced gynaecologists after a training period on the new catheter performing a minimum number of 50 mock transfers. Both catheters are maintained at +37° C. under sterile conditions. Immediately prior to embryo loading, the chosen catheter is flushed twice with the culture medium (Global, LifeGlobal, IVFonline, USA). With the new catheter 1 cm of air was first aspirated, then 0.5-1 cm of medium (approx 6-12 μl) containing the embryos followed by 2 cm of air. (The values in cm are referred to the aforesaid ink marks 6, i.e. the value of the stroke of the plunger in the cylindrical body of the catheter 1). Similarly, approx 1 cm of air was aspirated with the Tomcat catheter; the medium containing the embryos was then aspirated followed by approx 2 cm of air.

Immediately before embryo loading, the patient was prepared by gently inserting a bivalve speculum, careful aspiration of all the visible mucus and delicate wiping with a swab dampened with culture medium. Both uterine angle and position were confirmed or re-evaluated and, if considered necessary, the catheter was moulded to facilitate the overcoming of the internal os. Patients with an anteverted uterus were asked to fill their bladder in order to reduce the uterine angle. In the case of the new catheter 1, the ring mark 12 was positioned on the basis of previous hysterometry or ultrasound measurement of the cavity and the insertion of the catheter was arrested when the ring reached the external os to be sure that the tip of the catheter did not touch the uterine fundus. The position was confirmed by transabdominal ultrasonography. The embryos were then released by gentle pressure on the plunger, the catheter slowly withdrawn and immediately checked under the stereomicroscope for any retained embryos. The patient was then left on the bed to rest for 10 to 30 minutes, depending on the duration of the transfer and on any difficulties encountered. Easy transfer: the catheter reaches the cavity without any difficulties. Partially difficult transfer: some difficulties are encountered during negotiation of the internal os and the time of positioning the catheter into the cavity was delayed. Difficult transfer: considerable time was taken to enter the uterus (tenaculum, new catheter).

During the study, two conditions have been considered:

A) a insulin syringe (1 ml) connected to a catheter of 0.4 mm of internal diameter, and
B) the new catheter 1 with a constant 0.4 mm internal diameter with a plunger inside. Relationships between pressure and velocity will be analysed in different simulated conditions.

Simulated analysis of the two systems.

The plunger of the insulin syringe has a diameter of 4.75 mm with a corresponding surface of 17.34 mm² (A) while the plunger inside the catheter has a diameter of 0.4 mm with a corresponding surface of 0.125 mm² (B). The immediate consequence of those data is that when both plungers move by 1 mm, A transport about 140 times more water (or other fluid) than B. If we continue to calculate 1 mm shift of A corresponds to a charge of 1.734×10⁻² grams applied on the plunger, while 1 mm shift of B corresponds to 1.25×10⁻⁴ grams. Those theoretical values clearly indicate the differences between the two conditions. Now, if a force of 0.50 Newton (approx 50 gr), representing the minimal force able to move the plunger, is applied to the situation A (state of the art), the consequence is that 1 gr of water is ejected in 55 sec. which corresponds to a velocity of 0.018 gr/sec. On the other hand, if a force of 0.40 N (approx 40 gr) is applied to the plunger B (present invention), we obtain that 1 gr of water is ejected in 3300 sec with an ejection speed of 3.03×10⁻⁴ gr/sec i.e. 60 times less than A. If the maximum force (500 gr=5 N) is applied in the two conditions, A and B, the velocity increases dramatically in the situation A (state of the art), while don't significantly changes in situation B (present invention).

Regarding to the clinical results, no difficulties have been encountered during embryo loading facilitated by the semi-rigid structure of the new catheter, which reduces the fine movement of the tip under the stereomicroscope. Moreover, the transparency of the catheter and the marks on the plunger make positioning of the embryos easy. In 10 patients belonging to the Tomcat group, but in none of the new catheter group, after the transfer a small amount of blood was found on the catheter. There were no differences in the duration of the transfer and a tenaculum was never necessary.

Another advantage of the invention is that the tip of the new catheter was really well visible at ultrasound while the Tomcat was not always visible. Moreover, the ring positioned on the bases of the hysterometric measure, permits with the new catheter, a correct positioning of the embryos and avoid to touch the uterine fundus.

Moreover, the idea of the catheter of the present invention, was based on the observation that all the available catheters need a connection for a syringe to aspirate the embryos from culture medium into the catheter and to eject them into the uterine cavity; this imply a number of variable factors which are difficult to standardize. To limit the syringe-dependent problems some modifications have been done; for example some syringes have an end-course block to avoid the re-aspiration of the embryos into the catheter after the transfer but if the classical insulin syringe is used, the pressure on the plunger must be maintained until the complete withdrawal of the catheter from the uterine cavity. The diameter of the catheter contributes to realize a negative pressure when the catheter is withdrawn from the cavity; indeed when the catheter is withdrawn from the uterus it probably behaved like a plunger into a syringe considering that the endometrium adhere to the external wall of the catheter. With this in mind we think that a thinner catheter will probably realize less negative pressure than a thicker one.

Concerning the diameter of the syringe it is easy to note that it is up to 10 times higher than that of the catheter. This means that the movement of the plunger during the aspiration/ejection steps, causes an important increase of the speed of the embryos which is related with both the pressure applied on the plunger and the difference between the internal diameters of the syringe and of the catheter. In some conditions the catheter connected to the syringe (state of the art) determines a speed of the embryos very high, that is 80 Km/h=22000 mm/sec, while the new transfer procedure produces an ejection speed of 2 cm/sec=20 mm/sec (about 1000 times less than the state of the art procedure). The consequence is that the impact with the endometrial mucosa has a 10⁴ of difference. With the new catheter the variable of the pressure exerted on the plunger both by the same operator and by different operators is avoided. In fact, the embryo-ejection speed becomes an operator-independent variable. This becomes possible because the inner diameter of the catheter and that of the plunger are the same; in other words, when we move the plunger at a given velocity we are sure that the embryos move at that same velocity inside the catheter. With this new catheter, the embryos cannot be “fired” into the uterine cavity. As a matter of fact, high-speed ejection can be a potential source of embryo damage, increased risk of tubal pregnancy or loss of embryos outside the uterus. This last point represents a very important issue in the embryo transfer procedure and it is possible to speculate that the amount of transferred medium, the diameter of the catheter and the ejection speed constitute a few of the negative factors affecting the pregnancy rate. The echogenicity of the catheter tip was really good permitting to follow the entering of the catheter into the uterus and the correct positioning of the tip before the embryo release. With this catheter the operator can chose to perform the transfer simply helped by the ring position on the catheter or in an ultrasound-guided way or both associated. Moreover, the new catheter seems to function with a limited amount of trauma; in fact, blood was never found on the catheter after the transfer. The soft distal part, the reduced external diameter joint, together with the skill of the operator in both negotiating the internal os and entering the uterine cavity, are probably the main reasons for the absence of blood on the catheter. The higher implantation and multiple pregnancy rates observed in the new group could be the direct consequence of both reduced endometrial disruption and possible decreased loss of embryos outside the uterus, so that all the transferred embryos can have a chance to implant.

Another advantage of the present invention is that the device for the embryo transfer is formed by a cylindrical hollow single body, without any connection, while in the state of the art it is necessary the connection between the syringe and the catheter.

While the invention has been described with respect to a specific embodiment thereof, it will be understood by those skilled in the art that variations and modifications may be made without departing from the essential features thereof.

What is claimed is:

1. A method for the intrauterine embryo transfer, according to a procedure in which the embryos are aspirated from a culture medium for being ejected into the uterine cavity, wherein the aspiration and the release of the embryos is realized using a catheter having a substantially constant inner diameter in which is inserted a relevant plunger.

2. The method of claim 1 wherein in the catheter has an inner diameter of 0.4 mm and wherein in the catheter are first aspirated air for a stroke of 1 cm, then medium containing the embryos, for a stroke of 0.5-1 cm of medium, corresponding approximately to 6-12 μl, followed by a stroke of 2 cm of air.

3. The method of claim 1 wherein a ring mark is fitted on the catheter on the basis of previous hysterometry or ultrasound measurement of the cavity and wherein the insertion of the catheter is arrested when the ring reaches the external os so as to the tip of the catheter do not touch the uterine fundus.

4. The method of claim 1 wherein the catheter is provided with an echogenic distal tip.

5. The method of claim 1 wherein the catheter is composed of:
a rigid proximal tube with an external diameter comprised between 4 and 8 Fr, having a length comprised between 70 and 150 mm;
a semi-rigid medial tube with external diameter comprised between 2 and 6 Fr, having a length comprised between 100 and 180 mm; and
a soft distal portion with external diameter comprised between 2 and 6 Fr, having a length comprised between 6 and 14 mm.

6. The method of claim 1 wherein the inner diameter of the catheter is about 0.4 mm.

7. A device for the intrauterine embryo transfer, according to a procedure in which the embryos are aspirated by means of a plunger-device from a culture medium for being ejected into the uterine cavity, wherein the device is composed by a tubular hollow single body or catheter, having a substantially constant inner diameter, and a plunger suitable to be directly inserted in said catheter.

8. The device of claim 7 wherein the inner diameter of the catheter is comprised between 0.1 and 1 mm.

9. The device of claim 7 wherein the inner diameter of the catheter is about 0.4 mm.

10. The device of claim 7 wherein the catheter is composed of:
a rigid proximal tube with an external diameter comprised between 4 and 8 Fr, having a length comprised between 70 and 150 mm;
a semi-rigid medial tube with external diameter comprised between 2 and 6 Fr, having a length comprised between 100 and 180 mm; and
a soft distal portion with external diameter comprised between 2 and 6 Fr, having a length comprised between 6 and 14 mm.

11. The device of claim 7 wherein the catheter is composed of:
a rigid proximal tube with an external diameter of about 6 Fr (2.00 mm), having a length of about 110 mm;
a semi-rigid medial tube with external diameter of about 4 Fr (1.33 mm), having a length of about 140 mm; and
a soft distal portion with external diameter of about 4 Fr (1.33 mm), having a length of about 10 mm.

12. The device of claim 7 wherein the catheter has an echogenic distal tip.

13. The device of claim 7 wherein the total length of the catheter is comprised between 180 and 300 mm.

14. The device of claim 7 wherein the total length of the catheter is about 240 mm.

15. The device of claim 7 wherein ink marks are placed on the catheter, in correspondence of its proximal and central part.

16. The device of claim 7 wherein ink marks are placed on the proximal part of the plunger and on the catheter, in correspondence of its proximal and central part.
